(19) United States
(12) Patent Application Publication
(10) Pub. No.: US 2018/0225947 A1
(43) Pub. Date: Aug. 9, 2018

(54) METHODS AND SYSTEMS FOR NON-INVASIVE MONITORING

(71) Applicant: Hubble Connected India Private Limited, Bangalore (IN)

(72) Inventors: Arzad Alam KHERANI, Bangalore (IN); Perumal Raj Sivarajan, Bangalore (IN); Balaji Chegu, Bangalore (IN); Ochintya Sharma, Bangalore (IN)

(73) Assignee: Hubble Connected India Private Limited, Bangalore (IN)

(21) Appl. No.: 15/947,966

(22) Filed: Apr. 9, 2018

(30) Foreign Application Priority Data
Apr. 10, 2017 (IN) 201741012806

(51) Int. Cl. G08B 21/18; A61B 5/00; G06K 9/00

(52) U.S. Cl. CPC G08B 21/182; G06K 9/00369; A61B 5/4815

(57) ABSTRACT

The embodiments herein disclose methods and systems for non-invasive monitoring of a subject for coverage by a cover, a method includes capturing at least one image of an environment comprising of the subject for monitoring. Further, the method includes identifying at least one region of interest on receiving the at least one image of the environment wherein the at least one region of interest includes the subject, the cover and a reference frame. Further, the method includes performing image segmentation on the identified region of interest to estimate exposed fraction of a body of the subject. The image segmentation is performed using a reference guided region growing mechanism which receives the learned features of the subject, the cover and the reference frame as inputs. Further, the method includes generating at least one alert indication to at least one user based on the exposed fraction of the body of the subject.

### CROSS REFERENCE TO RELATED APPLICATION

This application is based on and derives the benefit of Indian Provisional Application 201741012806 filed on 10 Apr. 2017, the contents of which are incorporated herein by reference.

### TECHNICAL FIELD

Embodiments disclosed herein relate to subject monitoring and more particularly to non-invasive monitoring of a subject for coverage by a cover.

### BACKGROUND

Current solutions to monitor whether a cover adequately covers a subject (wherein the cover can be at least one of a sheet, blanket, quilt, bed linens, shawl, or any means that can cover a subject) require expensive solutions, wherein the monitoring solutions are typically built into the cover. The cover with the solutions can monitor how much of the subject is covered by the cover. However, covers with inbuilt solutions may be expensive and it may not be able to purchase adequate number of such covers.

### OBJECTS

The principal object of embodiments herein is to disclose methods for non-invasive monitoring of a subject for coverage by a cover, wherein a camera monitors the subject.

These and other aspects of the embodiments herein will be better appreciated and understood when considered in conjunction with the following description and the accompanying drawings. It should be understood, however, that the following descriptions, while indicating at least one embodiment and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF FIGURES

Embodiments herein are illustrated in the accompanying drawings, through out which like reference letters indicate corresponding parts in the various figures. The embodiments herein will be better understood from the following description with reference to the drawings, in which:

FIGS. 1a and 1b depict a non-invasive system for monitoring a subject for coverage by a cover, according to embodiments as disclosed herein;

FIG. 2 depicts a block diagram illustrating various units of a monitoring engine for monitoring a subject for coverage by a cover, according to embodiments as disclosed herein;

FIG. 3 depicts a flow diagram illustrating a method for non-invasive monitoring of a subject for coverage by a cover, according to embodiments as disclosed herein;

FIG. 4 depicts an example diagram illustrating learning of cover related features for cover segmentation, according to embodiments as disclosed herein;

FIG. 5 depicts an example diagram illustrating detection of a body of a subject, a cover segmentation and a reference frame segmentation, according to embodiments as disclosed herein;

FIG. 6 depicts an example diagram illustrating image segmentation performed for estimating an exposed fraction of a subject, according to embodiments as disclosed herein; and

FIG. 7 depicts an example diagram illustrating generation of an alert for a user based on an estimated exposed level of a subject, according to embodiments as disclosed herein.

### DETAILED DESCRIPTION

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

The embodiments herein provide methods and systems for non-invasive monitoring of a subject for coverage by a cover.

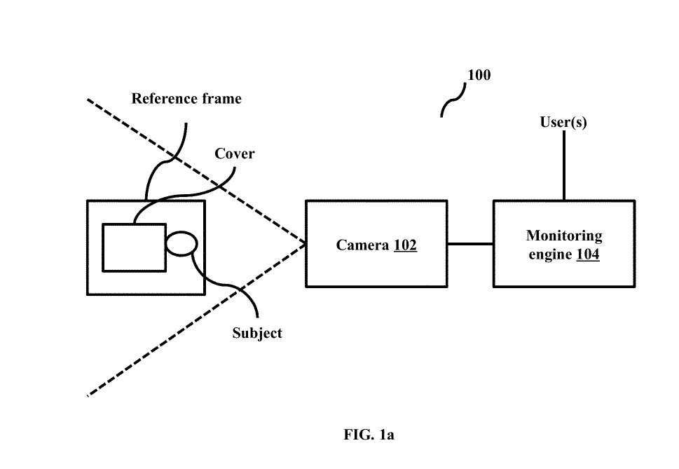

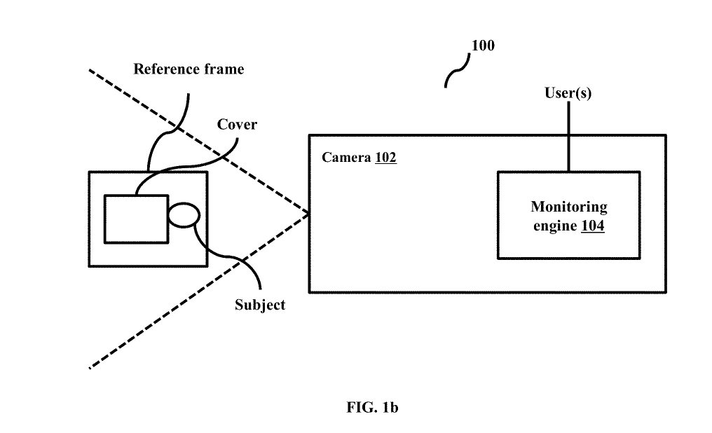

A method disclosed herein includes capturing one or more images of an environment comprising of the subject for monitoring. On receiving the one or more images of the environment, the method includes identifying one or more regions of interest. The one or more regions of interest may include the subject, the cover and a reference frame. Further, the method includes performing image segmentation on the identified one or more regions of interest to estimate an exposed fraction of a body of the subject. The image segmentation can be performed using a reference guided region growing mechanism. The reference guided region growing mechanism can use learned features of the subject, the cover and the reference frame to detect the body of the subject and perform cover segmentation and reference frame segmentation. Further, the method includes generating one or more alert indications to at least one user based on the estimated exposed fraction of the body of the subject. Referring now to the drawings, and more particularly to FIGS. 1a through 7, where similar reference characters denote corresponding features consistently throughout the figures, there are shown embodiments.

FIGS. 1a and 1b depict a non-invasive system 100 for monitoring a subject for coverage by a cover, according to embodiments as disclosed herein. The subject herein refers to a person who is being covered by the cover. Examples of the subject can be, but not limited to, a baby, a patient, a pet, a person under observation, and so on. The cover as referred to herein can be at least one of a sheet, blanket, quilt, bed linens, shawl, or any means that can be used to cover the subject. The cover can also comprise of more than cover, where more than one cover can be used to cover the subject.

As illustrated in FIGS. 1a and 1b, the non-invasive system 100 includes a camera 102 and a monitoring engine 104. At least one subject can be present in the field of view of the camera 102. The monitoring engine 104 can be at least one of a dedicated server, the cloud, a user device (such as a mobile phone, tablet, computer, laptop, Internet of Things (IoT) device, wearable devices, camera, and so on), and so on. In an embodiment herein, the monitoring engine 104 can use additional sensors such as, but not limited to, door sensors, motion sensors, thermometers, microphones, proximity sensors, and so on. The monitoring engine 104 can be connected to a user, wherein the monitoring engine 104 can enable the user to perform configuration(s). The user herein can be a person, who receives alerts from the monitoring engine 104. The same user or any other authorized user and/or entity may configure the alerts, as required. In an embodiment herein, more than one person can receive the alerts. In an embodiment, the camera 102 can perform all or some of the functions as performed by the monitoring engine 104 (as depicted in FIG. 1b).

Initially, the user can register the subject, the cover and the reference frame with the monitoring engine 104 using the camera 102 or any other device (such as a mobile phone, tablet, computer, laptop, Internet of Things (IoT) device, wearable devices, camera, and so on). Examples of the reference frame can be, but is not limited to, a bed, cot, couch, crib, and so on. Based on user registered inputs, the monitoring engine 104 learns features of the subject, the cover and the reference frame.

In an embodiment herein, the monitoring engine 104 can enable the camera 102 to fetch images of the reference frame and vicinity. The monitoring engine 104 provides the fetched images to the user to select relevant images for learning the features of the subject, the cover and the reference frame.

For monitoring the subject for coverage by the cover, the camera 102 captures images of an environment (for example, a room, a bed, a crib, a mat, a bed spread, and so on) in which the subject is located. The camera 102 provides the images to the monitoring engine 104. In an embodiment, the subject can be detected in the environment using the sensors that may be present in the environment such as door sensor, proximity sensors and so on.

On receiving the images from the camera 102, the monitoring engine 104 can be configured to identify a region of interest. The region of interest can include the subject, the cover and the reference frame. After identifying the region of interest, the monitoring engine 104 performs image segmentation on the region of interest to determine percentage of the subject covered by the cover. The image segmentation can be performed using the learned features of the subject, the cover and the reference frame. Further, the monitoring engine 104 compares the identified percentage of the subject covered by the cover with a pre-defined threshold.

Based on the comparison, the monitoring engine 104 generates alerts for the at least one user. The alerts can be provided at pre-defined intervals or on pre-defined events occurring (such as the cover slipping beyond a pre-defined percentage). The monitoring engine 104 can also provide alerts to the user, based on the configurations performed by the user. Further, the monitoring engine 104 can configure the alerts based on additional parameters such as room temperature, shivering of the subject, noises made by the subject, movement of the subject, and so on.

In an embodiment, the additional parameters can be detected using data received from door sensors, motion sensors, thermometers, microphones, proximity sensors, and so on. The monitoring engine 104 can also determine additional parameters such as sleep quality metric, sleep quality graphs, and so on, based on the received information related to the subject. In an embodiment, the monitoring engine 104 receives information related to the subject from the camera 102, an image database, a backend system and so on.

In an embodiment herein, the monitoring engine 104 can use a suitable means such as rate limit analysis to optimally use battery, and computation power.

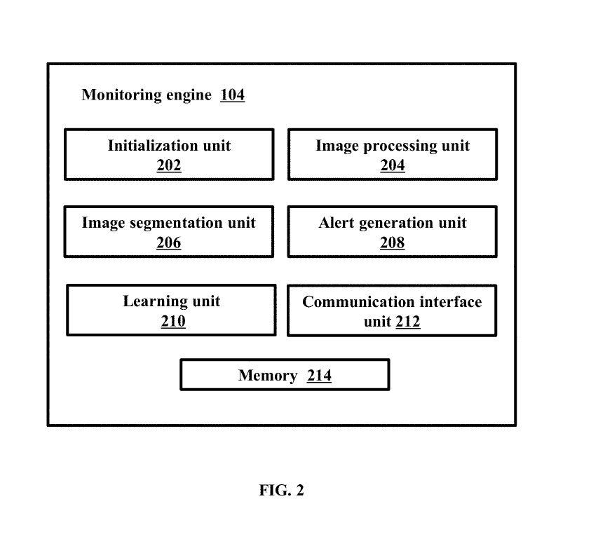

FIG. 2 depicts a block diagram illustrating various units of the monitoring engine 104 for monitoring the subject for coverage by the cover, according to embodiments as disclosed herein. The monitoring engine 104 can detect the exposed fraction of the subject being monitored by the camera 102 and alerts the user based on the detected exposed fraction of the subject. The monitoring engine 104 includes an initialization unit 202, an image processing unit 204, an image segmentation unit 206, an alert generation unit 208, a learning unit 210, a communication interface unit 212 and a memory 214.

The initialization unit 202 can be configured to receive inputs from the user through a registration process, which is initiated by the user. The inputs include information/images related to the subject, the cover and the reference frame. The user can use the camera 102 or any other device (such as a mobile phone, tablet, computer, laptop, Internet of Things (IoT) device, wearable devices, camera, and so on) for registration.

In absence of user inputs, the initialization unit 202 enables the camera 102 to capture images of the reference frame and vicinity. For example, the images may comprise of the cover, the subject with the cover, the subject without the cover and so on. Images as referred to herein may be at least one of, but not limited to, a picture, a video, one or more frames from a video, an animation, and so on. Further, the initialization unit 202 provides the images to the user and obtains feedback by allowing the user to select most relevant images. The initialization unit 202 can store the images selected by the user in a suitable location such as, but not limited to, memory 214, an image database, a server, the Cloud, or the like. In an embodiment herein, the initialization unit 202 can re-size and transform the images to save storage space and performance.

The initialization unit 202 can be further configured to process at least one image related to the subject, the cover and the reference frame. The at least one image can be, but is not limited to, an image registered by a user, a most relevant image selected by the user, a previous image captured by the camera 102, a stored image and so on.

The initialization unit 202 processes the at least one image related to the subject to learn the features of the subject such as, but not limited to, eyes, lips, nose, arms, legs, shoulders, and so on. Further, the initialization unit 202 processes the at least one image related to the cover to learn the features of the cover. In order to learn the features of the cover, the initialization unit 202 derives a plurality of key identifiers or points for the cover (herein after referred to as seeds) that are clearly distinguishable and such that at least one of the seeds is always visible. The seeds herein can refer to the points identified on the cover. The seeds can be dynamic and added to or deleted from over time as per learning. Further, the initialization unit 202 can determine factors such as, but not limited to, location, shape, size and any other related factors of the cover using the identified points. In addition, the initialization unit 202 identifies boundaries of the cover using the seeds and their relative location with respect to the overall shape of the cover.

Similarly, the initialization unit 202 processes the at least one image related to the reference frame to learn the features of the reference frame. Examples of the reference frame can be, but not limited to, a room, a bed, a crib, a mat, a bed spread, and so on. In order to learn the features of the reference frame, the initialization unit 202 derives a plurality of key identifiers or seeds for the reference frame that are clearly distinguishable and such that at least one of the seeds is always visible. Further, the initialization unit 202 can determine factors such as, but not limited to, location, shape, size and any other related factors of the reference frame using the identified points. In addition, the initialization unit 202 identifies boundaries of the reference frame using the seeds and their relative location with respect to the overall shape of the reference frame.

The image processing unit 204 can be configured to receive at least one image of the environment from the camera 102. The environment may comprise the subject, which needs to be monitored. The image processing unit 204 may process the image to identify the region of interest by filtering noises and unnecessary activities present in the environment. The identified region of interest may include the subject, the cover and the reference frame. The image processing unit 204 also checks if there is movement in the region of interest by comparing the received image with previous images and/or reference images.

The image segmentation unit 206 can be configured to perform image segmentation on the region of interest identified by the image processing unit 204. The image segmentation can be performed to estimate an exposed fraction of the body of the subject. In an embodiment, the image segmentation unit 206 can perform the image segmentation using a reference guided region growing mechanism for the reference frame, the subject and the cover. The image segmentation unit 206 can use the learned features of the subject as inputs to the reference guided region growing mechanism for detecting the body of the subject. The image segmentation unit 206 can use the learned features of the cover (as learned by the initialization unit 202) as inputs to the reference guided region growing mechanism for cover segmentation. Similarly, the image segmentation unit 206 can use the learned features of the reference frame (as learned by the initialization unit 202) as inputs to the reference guided region growing mechanism for reference frame segmentation.

In an embodiment herein, the image segmentation unit 206 can consider depth as an additional parameter which is calculated from a depth camera for performing the image segmentation. The image segmentation unit 206 can also use additional information such as, but not limited to, reference data, images, transformed image data, classifications, and so on for performing the image segmentation. The image segmentation unit 206 provides information about the estimated exposed fraction of the body of the subject to the alert generation unit 208.

The alert generation unit 208 can be configured to compare the estimated exposed fraction of the subject with the pre-defined threshold. Based on the comparison, the alert generation unit 208 can raise the alert to the user. The alert can be in the form of at least one of an audio alert, a visual alert, and so on. The alert can be in the form of at least one of an email, a SMS (Short Messaging Service), a pop-up, a push notification, a widget, and so on. The alert can comprise of information such as a timestamp, the percentage of the subject covered, an image/screenshot of the subject, and so on.

The learning unit 210 can be configured to gather the feedback/reaction of the user to the alert and update the learned features. For example, the learning unit 210 can determine that the alert is a false positive, on determining that the user has pressed ignore or swipes off the alert and does nothing. For example, the learning unit 210 can determine that the alert is right, on determining that the user has moved to room or vicinity of the subject. In an embodiment herein, the learning unit 210 can gather explicit feedback at periodic intervals of time. The learning unit 210 determines movement of the user to the room by location change of a device carried by the user (if location details provided by the device are accurate enough) or based on door open event detected by the door sensor in the subject's room or based on motion/person detected by the camera in the subject's room. Other sensors (such as proximity sensor if any in subject's room) can also be used to detect if there was a response to alert.

The learning unit 210 can be further configured to update the learned features of the subject, the cover and the reference frame by updating the seeds in a continuous manner. The learning unit 210 can also learn and update the impact of various lighting conditions on the seeds and update the related segmentation aspects accordingly. In an embodiment herein, the learning unit 210 can update the reference images and other information, based on user feedback and responses.

The learning unit 210 further monitors the user for feedback, such as the user ignoring the alert (for example, by not taking any action, performing a pre-defined action to dismiss the alert, and so on), the user performing an action related to the alert (such as checking the subject, accessing the camera 102 to view the subject, and so on). The learning unit 210 can also gather feedback from the user explicitly, by requesting the user on infrequent basis. The learning unit 210 can use the feedback to improve the reference images, the seeds, and any other feature that improves the performance of the monitoring engine 104. The learning unit 210 can also enable analysis to be performed manually for chosen samples related to the subject, the cover and the reference frame. The learning unit 210 can modify the reference frame(s), based on the feedback.

The learning unit 210 can provide the feedback to a back-end system (such as a cloud, a file server, a data server, and so on). The back-end system can use the feedback to improve the reference images, seeds, and any other feature that improves the performance of the monitoring engine 104. The back-end system can also enable analysis to be performed manually for chosen samples. The back-end system can modify the reference frame(s), based on the feedback.

The communication interface unit 212 can be configured to establish communication with the camera 102.

The memory 214 can be configured to store user registered images, the images captured by the camera and the learned features for the subject, the cover and the reference frame. The memory 214 may include one or more computer-readable storage media. The memory 214 may include non-volatile storage elements. Examples of such non-volatile storage elements may include magnetic hard discs, optical discs, floppy discs, flash memories, or forms of electrically programmable memories (EPROM) or electrically erasable and programmable (EEPROM) memories. In addition, the memory 214 may, in some examples, be considered a non-transitory storage medium. The term “non-transitory” may indicate that the storage medium is not embodied in a carrier wave or a propagated signal. However, the term “non-transitory” should not be interpreted to mean that the memory 214 is non-movable. In some examples, the memory 214 can be configured to store larger amounts of information than the memory. In certain examples, a non-transitory storage medium may store data that can, over time, change (e.g., in Random Access Memory (RAM) or cache).

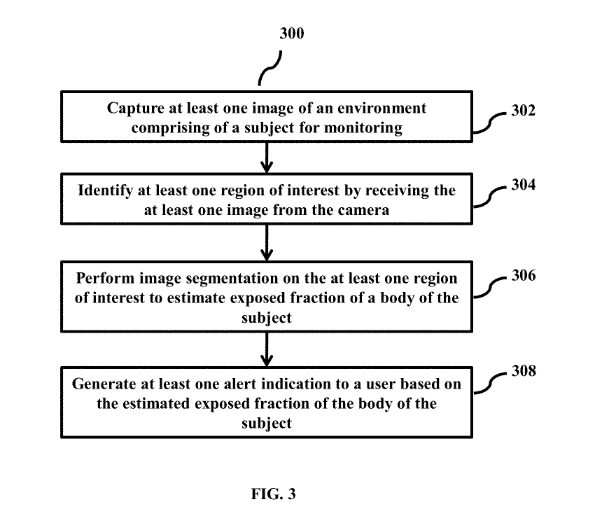

FIG. 3 depicts a flow diagram 300 illustrating a method for non-invasive monitoring of the subject for coverage by the cover, according to embodiments as disclosed herein.

At step 302, the method includes capturing the at least one image of the environment comprising of the subject for monitoring. The method allows the camera 102 to capture the at least one image of the environment comprising of the subject for monitoring.

At step 304, the method includes identifying the region of interest on receiving the at least one image from the camera 102. The method allows the image processing unit 204 of the monitoring engine 104 to identify the region of interest on receiving the at least one image from the camera 102. The region of interest includes the subject, the cover and the reference frame.

At step 306, the method includes performing image segmentation on the region of interest. The method allows the image segmentation unit 206 of the monitoring engine 104 to perform image segmentation on the region of interest. The image segmentation can be performed using the reference guided region growing mechanism for the subject, the cover and the reference frame.

The reference guided region growing mechanism can detect the body of the subject on receiving the learned features of the subject from the initialization unit 202. The reference guided region growing mechanism can perform the cover segmentation on receiving the learned features of the cover from the initialization unit 202. The learned features of the cover may include the boundaries of the cover detected using the seeds and their relative location with respect to the overall shape of the cover. Similarly, the reference guided region growing mechanism can perform the reference frame segmentation on receiving the learned features of the reference frame from the initialization unit 202. The learned features of the reference frame may include the boundaries of the reference frame detected using the seeds and their relative location with respect to the overall shape of the reference frame. Further, the initialization unit 202 learns the features of the subject, the cover and the reference frame by processing the at least one input image which include user registered image, the most relevant image selected by the user, the previous image captured by the camera 102, the stored image and so on.

At step 308, the method includes generating at least one alert indication for the at least one user based on the exposed fraction of the body of the subject. The method allows the alert generation unit 208 to generate the at least one alert indication for at least one user based on the exposed fraction of the body of the subject. The alert generation unit 208 compares the exposed fraction of the body of the subject with the pre-defined threshold and accordingly generates the at least one alert indication for the user. Thus, the subject can be monitored for coverage by the cover without involving any instruments/devices built into the cover.

The various actions, acts, blocks, steps, or the like in the method and the flow diagram 300 may be performed in the order presented, in a different order or simultaneously. Further, in some embodiments, some of the actions, acts, blocks, steps, or the like may be omitted, added, modified, skipped, or the like without departing from the scope of the invention.

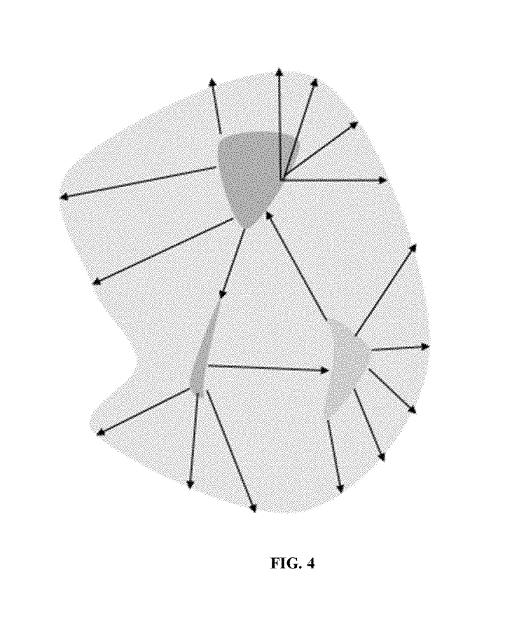

FIG. 4 depicts an example diagram illustrating learning of the cover related features for the cover segmentation, according to embodiments as disclosed herein. Embodiments herein enable the monitoring engine 104 to learn the features of the cover by receiving at least one input related to the cover. The learned features of the cover can be used for the cover segmentation. For example, the at least one input can include the user registered image related to the cover. On receiving the user registered image related to the cover, the monitoring engine 104 learns the features of the cover by deriving the seeds for the cover and estimating the parameters related to the cover such as location, size, shape and so on. The seeds can be uniquely identifiable regions (key identifiers) on the cover. Further, the seeds and their relative location with respect to the overall shape help in identifying the boundaries of the cover and further help in the cover segmentation.

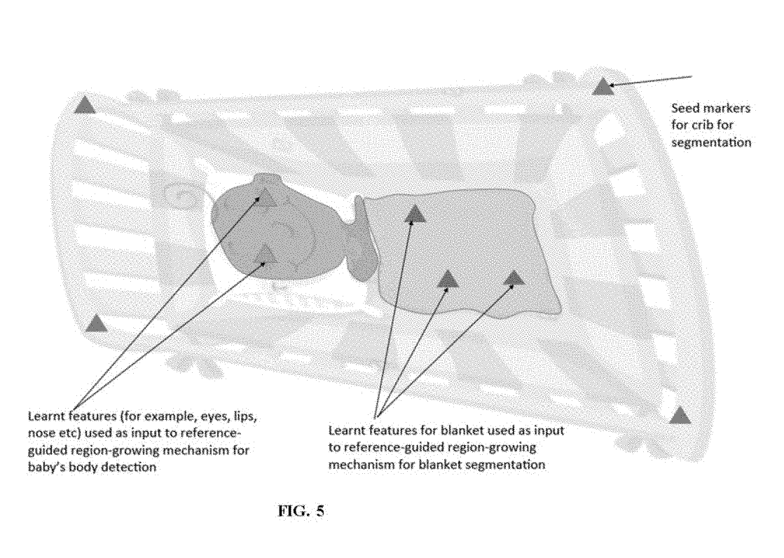

FIG. 5 depicts an example diagram illustrating detection of the body of the subject, the cover segmentation and the reference frame segmentation, according to embodiments as disclosed herein. Consider a baby sleeping in a crib, which needs to be monitored for coverage by blanket as illustrated in FIG. 5. The monitoring engine 104 can identify the region of interest on receiving at least one image from the camera 102. The region of interest may include the baby, the blanket and the crib. After identifying the region of interest, the monitoring engine 104 uses the reference guided region growing mechanism to detect a body of the baby. For detecting the body of the baby, the reference guided region growing mechanism uses the learned features (nose, eyes, lips and so on) of the baby. For example, the features of the baby can be learned initially using at least one user registered image related to the baby.

Further, the monitoring engine 104 uses the reference guided region growing mechanism to perform blanket segmentation. For the blanket segmentation, the reference guided region growing mechanism uses learned features of the blanket that include derived seeds for the blanket. Similarly, the monitoring engine 104 uses the reference guided region growing mechanism to perform crib segmentation. For the crib segmentation, the reference guided region growing mechanism uses learned features of the crib that include seed markers derived for the crib. For example, the features of the blanket and the crib can be learned initially using at least one user registered image related to the blanket and the crib.

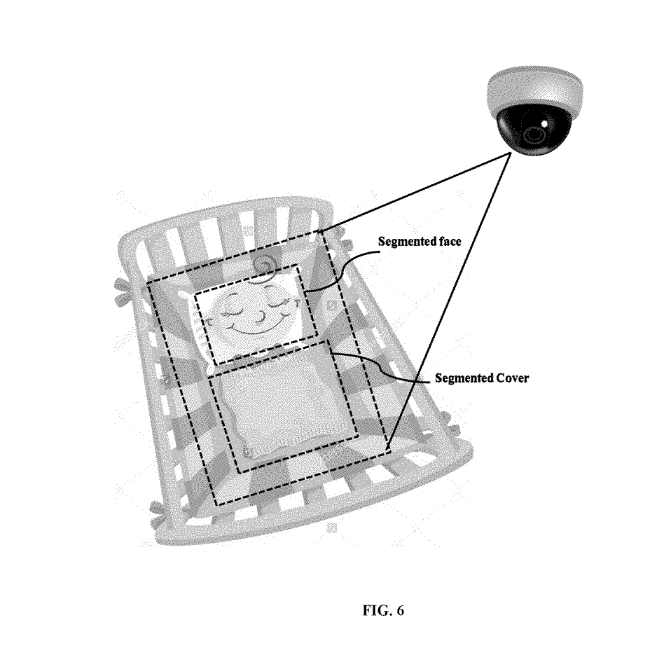

FIG. 6 depicts an example diagram illustrating the image segmentation performed for estimating the exposed fraction of the subject, according to embodiments as disclosed herein. Consider a baby sleeping in a crib, which needs to be monitored for coverage by blanket as illustrated in FIG. 6. The monitoring engine 104 receives at least one image captured by the camera 102 and identifies the region of interest. For example, the region of interest may include the baby with the blanket. After identifying the region of interest, the monitoring engine 104 detects face of the baby using the reference guided region growing mechanism. Similarly, the monitoring engine 104 performs blanket segmentation using the reference guided region growing mechanism. Thus, the detection of the face and body of the baby and the blanket segmentation helps in detecting the exposed level of the body of the baby.

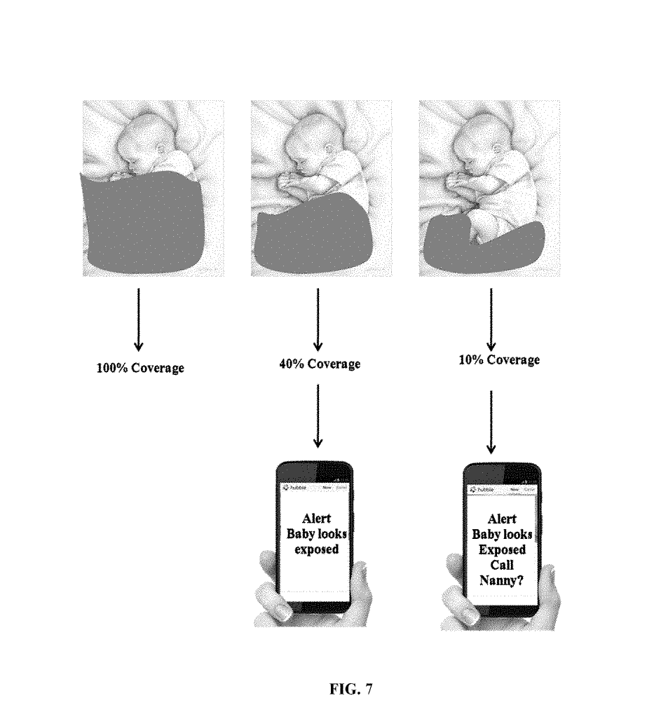

FIG. 7 depicts an example diagram illustrating generation of the alert for the user based on the estimated exposed level of the subject, according to embodiments as disclosed herein. Embodiments herein generate the alert for the user by comparing the estimated exposed level of the subject with the pre-defined threshold. For example, on determining that 100% of the baby's body is covered with the blanket, the monitoring engine 104 does not generate any alert for the user. On determining that 40% of the baby's body is covered with the blanket, the monitoring engine 104 sends the alert to the user by providing information about the percentage of the coverage. On determining that 10% of the baby's body is covered with the blanket, the monitoring engine 104 sends the alert to the user to inform the caretaker.

The embodiments disclosed herein can be implemented through at least one software program running on at least one hardware device and performing network management functions to control the network elements. The network elements shown in FIG. 1 and FIG. 2 include blocks, which can be at least one of a hardware device, or a combination of hardware device and software module.

The embodiments disclosed herein describe non-invasive methods and systems for monitoring a subject for coverage by a cover, wherein the system uses at least one camera. Therefore, it is understood that the scope of the protection is extended to such a program and in addition to a computer readable means having a message therein, such computer readable storage means contain program code means for implementation of one or more steps of the method, when the program runs on a server or mobile device or any suitable programmable device. The method is implemented in a preferred embodiment through or together with a software program written in e.g. Very high speed integrated circuit Hardware Description Language (VHDL) another programming language, or implemented by one or more VHDL or several software modules being executed on at least one hardware device. The hardware device can be any kind of portable device that can be programmed. The device may also include means which could be e.g. hardware means like e.g. an ASIC, or a combination of hardware and software means, e.g. an ASIC and an FPGA, or at least one microprocessor and at least one memory with software modules located therein. The method embodiments described herein could be implemented partly in hardware and partly in software. Alternatively, the invention may be implemented on different hardware devices, e.g. using a plurality of CPUs.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the embodiments as described herein.

We claim:

1. A method for non-invasive monitoring of a subject for coverage by a cover, the method comprising:
capturing, by a camera (102), at least one image of an environment comprising of the subject for monitoring;
identifying, by a monitoring engine (104), at least one region of interest on receiving the at least one image of the environment from the camera (102), wherein the at least one region of interest includes the subject, the cover and a reference frame;
performing, by the monitoring engine (104), image segmentation on the at least one region of interest to estimate exposed fraction of a body of the subject, wherein the image segmentation is performed using a reference guided region growing mechanism; and
generating, by the monitoring engine (104), at least one alert indication to at least one user based on the exposed fraction of the body of the subject.

2. The method of claim 1, further comprising:
receiving, by the monitoring engine (104), at least one input image related to the subject, the cover and the reference frame;
learning, by the monitoring engine (104), at least one feature of the subject in response to receiving the at least one input image related to the subject;
learning, by the monitoring engine (104), at least one feature of the cover in response to receiving the at least one input image related to the cover, wherein learning the at least one feature of the cover includes
deriving a set of seeds for the cover to determine at least one parameter related to the cover, wherein the set of seeds represent a plurality of key identifiers for the cover and the at least one parameter includes at least one of location, shape and size; and
identifying boundaries of the cover using the set of seeds and corresponding location with respect to the shape of the cover; and
learning, by the monitoring engine (104), at least one feature of the reference frame in response to receiving the at least one input image related to the reference frame, wherein learning the at least one feature of the reference frame includes
deriving a set of seeds for the reference frame to determine at least one parameter related to the reference frame, wherein the set of seeds represent a plurality of key identifiers for the reference frame and the at least one parameter includes at least one of location, shape and size; and
identifying boundaries of the reference frame using the set of seeds and corresponding location with respect to the shape of the reference frame.

3. The method of claim 2, wherein the at least one input image related to the subject, the cover and the reference frame includes at least one of at least one user registered input, at least one previous image captured by the camera (102) and at least one stored image.

4. The method of claim 1, wherein performing image segmentation using the reference guided region growing mechanism includes
detecting the body of the subject using the learned at least one feature of the subject;
performing cover segmentation using the learned at least one feature of the cover; and
performing reference frame segmentation using the learned at least one feature of the reference frame.

5. The method of claim 1, further comprising receiving feedback, by the monitoring engine (104), from the at least one user for the at least one alert indication for updating the at least one feature of the subject, the cover and the reference frame.

6. The method of claim 1, further comprising configuring, by the monitoring engine (104), the at least one alert indication based on at least one of room temperature, shivering of the subject, noises made by the subject and continuous movement of the subject.

7. The method of claim 1, further comprising determining, by the monitoring engine (104), at least one additional parameter including at least one of sleep quality metrics and sleep quality graphs related to the subject.

8. A system (100) for performing non-invasive monitoring of a subject for coverage by a cover, the system (100) comprises:
a camera (102) configured to
capture at least one image of an environment comprising of the subject for monitoring; and
a monitoring engine (104) connected to the camera (102), wherein the monitoring engine (104) comprises:
an image processing unit (204) configured to identify at least one region of interest on receiving the at least one image from the camera (102), wherein the at least one region of interest includes the subject, the cover and a reference frame;
an image segmentation unit (206) configured to perform image segmentation on the at least one region of interest to estimate exposed fraction of a body of the subject, wherein the image segmentation is performed using a reference guided region growing mechanism; and
an alert generation unit (208) configured to generate at least one alert indication to at least one user based on the estimated exposed fraction of the body of the subject.

9. The system (100) of claim 8, wherein the monitoring engine (104) further comprises an initialization unit (202) configured to:
receive at least one input image related to the subject, the cover and the reference frame;
learn at least one feature of the subject in response to receiving the at least one input image related to the subject;
learn at least one feature of the cover in response to receiving the at least one input image related to the cover by
deriving a set of seeds for the cover to determine at least one parameter related to the cover, wherein the set of seeds represent a plurality of key identifiers for the cover and the at least one parameter includes at least one of location, shape and size; and
identifying boundaries of the cover using the set of seeds and corresponding location with respect to the shape of the cover; and
learn at least one feature of the reference frame in response to receiving the at least one input image related to the reference frame by
deriving a set of seeds for the reference frame to determine at least one parameter related to the reference frame, wherein the set of seeds represent a plurality of key identifiers for the reference frame and the at least one parameter includes at least one of location, shape and size; and
identifying boundaries of the reference frame using the set of seeds and corresponding location with respect to the shape of the reference frame.

10. The system (100) of claim 9, wherein the at least one input image related to the subject, the cover and the reference frame includes at least one of at least one user registered input, at least one previous image captured by the camera (102) and at least one stored image.

11. The system (100) of claim 8, wherein the image segmentation unit (206) is further configured to
detect the body of the subject using the learned at least one feature of the subject;
perform cover segmentation using the learned at least one feature of the cover; and
perform reference frame segmentation using the learned at least one feature of the reference frame.

12. The system (100) of claim 8, wherein the monitoring engine (104) further comprises a learning unit (210) to receive feedback from the at least one user for the at least one alert indication to update the at least one feature of the subject, the cover and the reference frame.

13. The system (100) of claim 8, wherein the monitoring engine (104) is further configured to configure the at least one alert indication based on at least one of room temperature, shivering of the subject, noises made by the subject and continuous movement of the subject.

14. The system (100) of claim 8, wherein the monitoring engine (104) is further configured to determine at least one additional parameter including at least one of sleep quality metrics and sleep quality graphs related to the subject.
